(19) United States
(12) Patent Application Publication
(10) Pub. No.: US 2023/0253081 A1
(43) Pub. Date: Aug. 10, 2023

(54) METHOD FOR PROVIDING ELECTRONIC MEDICAL RECORDS FOR ANIMAL PATIENTS

(71) Applicant: PECO INC., Tokyo (JP)

(72) Inventor: Jun OKAZAKI, Tokyo (JP)

(21) Appl. No.: 17/907,646

(22) PCT Filed: Mar. 30, 2020
(86) PCT No.: PCT/JP2020/014520

§ 371 Date: Apr. 3, 2023

(51) Int. Cl. G16H 10/60; G16H 20/10

(52) U.S. Cl. CPC G16H 10/60; G16H 20/10

(57) ABSTRACT

An electronic medical chart information related to an animal patient to a user. A method for providing an electronic medical chart related to an animal patient, the method comprising: managing medical chart information, including prescription drug information regarding prescription drug contents and medication information for an animal patient, and transmitting prescription drug information to the animal patient terminal of the animal patient, wherein the prescription drug information includes contents related to medication methods.

### TECHNICAL FIELD

The present invention relates to a method for providing an electronic medical chart related to an animal patient that manages medical information for the animal patient.

### BACKGROUND ART

Conventionally, a method for retrieving information related to an electronic medical chart that manages medical information of a human patient has been disclosed.

For example, a method of retrieving a corresponding patient name using a disease name and progress information included in an electronic medical chart as a key, displaying a list of patient name, disease name, progress information, and consultation date in a retrieved results, and selecting the date of receipt to display the corresponding electronic medical chart is disclosed (Patent Literature 1).

### PRIOR ART LIST

### Patent Literature

Patent Literature 1: Japanese Unexamined Patent Publication No. 2013-140604

### SUMMARY OF THE INVENTION

### Technical Problem

However, Patent Literature 1 is a retrieval system provided for hospitals, although it can retrieve medical chart data related to medical history data from a medical chart database based on patient information and date information in the medical history data retrieved from the medical history database and is not designed for users such as owners of animal patients.

Therefore, it is an object of the present invention to provide electronic medical chart information related to an animal patient to a user, particularly to provide a method capable of easily administering medication.

### Technical Solution

According to an embodiment of the present invention, there is provided a method for providing an electronic medical chart related to an animal patient, the method comprising: managing medical chart information, including prescription drug information regarding prescription drug contents and medication information for an animal patient; and transmitting prescription drug information to the animal patient terminal of the animal patient, wherein the prescription drug information includes contents related to medication methods.

### Advantageous Effects

According to the present invention, electronic medical chart information related to an animal patient can be provided to a user, and in particular, the user can conveniently administer medication at a place rather than a hospital such as home.

### BRIEF DESCRIPTION OF THE DRAWING

FIG. 1 is a block configuration diagram showing an electronic medical chart providing system according to a first embodiment of the present invention;

FIG. 2 is a functional block configuration diagram of the server terminal 100 of FIG. 1;

FIG. 3 is a functional block configuration diagram of the animal hospital terminal 300 of FIG. 1;

FIG. 4 is a schematic diagram showing an example of electronic medical chart information stored in the server 100;

FIG. 5 is an example of a flowchart which explains a method for providing an electronic chart according to the first embodiment of the present invention;

FIG. 6 is an example of a chat user interface displayed on the user terminal 200 according to the first embodiment of the present invention;

FIG. 7 is another example of a chat user interface displayed on the user terminal 200 according to the first embodiment of the present invention; and

FIG. 8 is yet another example of a chat user interface displayed on the user terminal 200 according to the first embodiment of the present invention.

### DETAILED DESCRIPTION OF THE EMBODIMENTS

Hereinafter, embodiments of the present invention will be described with reference to the accompanying drawings. Further, the embodiments described below do not unreasonably limit the contents of the present invention described in the claims. Further, all components shown in the embodiments are not always essential components of the present invention

### Embodiment 1

### Configuration

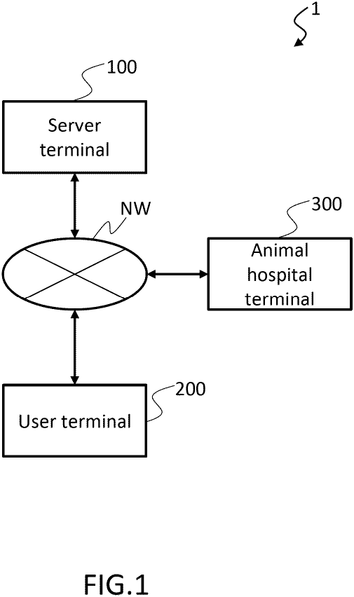

FIG. 1 is a block configuration diagram showing an electronic medical chart providing system 1 according to a first embodiment of the present invention. This electronic medical chart providing system 1 provides a user interface for viewing and/or editing an electronic medical chart, which is displayed via a display or the like of one or more animal hospital terminals.

The electronic medical chart providing system 1 has a server terminal (data processing device) 100, a user terminal 200, and a network NW. The server terminal 100 and the user terminal 200 are connected via a network NW. The network NW is comprised of the Internet, an intranet, a wireless LAN (Local Area Network), a WAN (Wide Area Network), and the like.

The server terminal 100 is a device that stores and processes electronic medical chart information for each animal patient user. For example, it may be a general-purpose computer such as a workstation or personal computer, or it may be logically realized by cloud computing.

The user terminal 200 is an information processing device such as a personal computer or a tablet terminal that is owned and used by the owner of an animal patient or the like, and may be configured by a smart phone, a mobile phone, a PDA, or the like.

The animal hospital terminal 300 is an information processing device such as a personal computer or a tablet terminal that is managed by the animal hospital and used by doctors, nurses, staff, and the like of the animal hospital, and may be configured by a smart phone, a mobile phone, a PDA, or the like.

In this embodiment, the electronic medical chart providing system 1 includes a server terminal 100, a user terminal 200 and an animal hospital terminal 300 and is described as a configuration for performing an operation on the server terminal 100 using the user terminal 200 and/or the animal hospital terminal 300. However, the server terminal 100 may be configured as a stand-alone, and the server terminal 100 itself may be provided with a function operated by the user. By installing all or a part of the programs executed in the server terminal 100 in the user terminal 200 and/or the animal hospital terminal 300, all or a part of the functions performed by the server terminal 100 can be performed by the user terminal 200 and/or the animal hospital terminal 300.

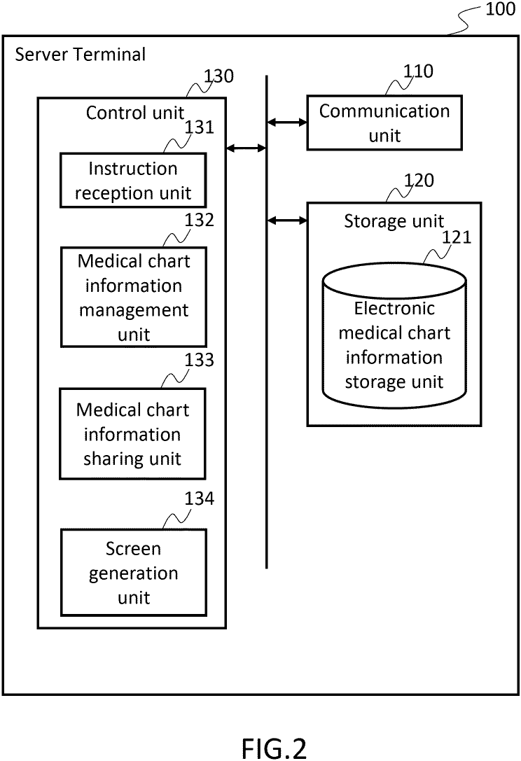

FIG. 2 is a functional block configuration diagram of the server terminal 100 of FIG. 1. The server terminal 100 includes a communication unit 110, a storage unit 120 and a control unit 130.

The communication unit 110 is a communication interface for communicating with the user terminal 200 and the animal hospital terminal 300 via the network NW, and for example, performs communication according to a communication protocol such as TCP/IP (Transmission Control Protocol/Internet Protocol).

The storage unit 120 stores programs executed by the control unit 130, input data or the like received from the user terminal 200 and/or the animal hospital terminal 300, and is comprised of RAM (Random Access Memory), ROM (Read Only Memory), HDD (Hard Disk Drive), etc. The storage unit 120 also has an electronic medical chart information storage unit 121 that stores electronic medical chart information for each user. Further, the storage unit 120 can store various data or programs including: (not shown) data for performing graphic representation such as icons that make up the electronic medical chart user interface, text data necessary for displaying each piece of information that makes up the electronic medical chart interface, layout determination data for determining the layout of the electronic chart interface, and a diagnostic information recommendation program for recommending diagnostic information based on input content. Further, a database (not shown) storing each data may be constructed outside the storage unit 120.

The control unit 130 executes a program stored in the storage unit 120 to thereby control the overall operation of the server terminal 100 and is comprised of a CPU (Central Processing Unit), a GPU (Graphics Processing Unit), and the like. The functions of the control unit 130 include an instruction reception unit 131 from the user terminal 120 of the user, a medical chart information management unit 132, a medical chart information sharing unit 133, and a screen generation unit 134. These are activated by programs stored in the storage unit 120 and executed by the server terminal 100, which is a computer (electronic computer).

The instruction reception unit 131 accepts instructions from the user terminal 200 and/or the animal hospital terminal 300 via the communication unit 110 when the user and/or the person in charge of the animal hospital make a predetermined request by making an input via a user interface such as a web screen displayed on the display of the user terminal 200 and/or the animal hospital terminal 300.

The medical chart information management unit 132 executes processing related to medical chart information based on a request received from a user and/or a person in charge of an animal hospital.

The medical chart information sharing unit 133 executes processing for sharing medical chart information with a user terminal.

The screen generation unit 133 generates screen information for configuring the user interface displayed on the user terminal 200 and/or the animal hospital terminal 300. For example, the image and text data stored in the storage unit 120 are used as materials, and various images and text are arranged in a predetermined area of the user interface based on predetermined layout rules, thereby generating the user interface. Processing related to the screen generation unit 133 can also be executed by a GPU (Graphics Processing Unit).

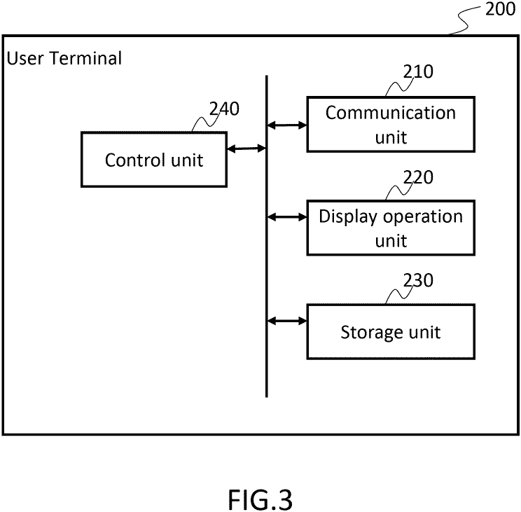

FIG. 3 is a functional block configuration diagram of the animal hospital terminal 300 of FIG. 1. The user terminal 200 includes a communication unit 310, a display operation unit 220, a storage unit 230, and a control unit 240.

The communication unit 210 is a communication interface for communicating with the server terminal 100 via the network NW, and for example, communication is performed according to a communication protocol such as TCP/IP.

The display operation unit 220 is a user interface used to input an instruction by a user and display text, images, etc. according to input data from the control unit 240, is comprised of a display, a keyboard and a mouse if the user terminal 200 is comprised of a personal computer, and is comprised of a touch panel or the like if the user terminal 200 is comprised of a tablet terminal. The display operation unit 220 is activated by a control program stored in the storage unit 230 and executed by the user terminal 200, which is a computer (electronic computer).

The storage unit 230 stores programs, input data, and the like for executing various control processes and functions in the control unit 340, and is comprised of RAM, ROM, and the like. Also, the storage unit 330 temporarily stores the communication content with the server terminal 100.

The control unit 240 controls the overall operation of the user terminal 200 by executing programs stored in the storage unit 230, and is comprised of a CPU, a GPU, and the like.

In this embodiment, since the transmission and reception of information between the server terminal 100 and the user terminal 200 will be mainly described, the description of the functional configuration of the animal hospital terminal 200 is omitted. The functional configuration of the animal hospital terminal 300 can be substantially equivalent to the functional configuration of the user terminal 200.

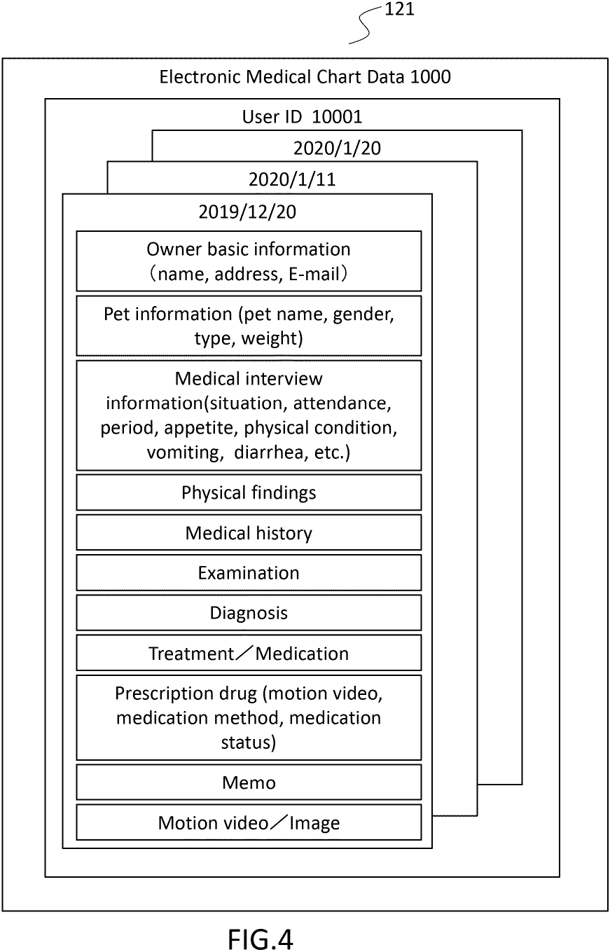

FIG. 4 is a schematic diagram showing an example of electronic medical chart information stored in the storage unit 120 of the server 100.

For example, the electronic medical chart information 1000 is managed in association with a user ID for each user, and the medical chart information for each user is managed for each medical examination reception date. Here, the user includes an animal patient. For example, the electronic medical chart information input on Aug. 20, 2019 as the medical examination reception date for the user identified by the user ID 10001 may include basic information of the animal patient’s owner (name, address, email, etc.), animal patient (pet) information (pet name, gender, type, weight, etc.), medical interview information (situation, attendance, period, appetite, physical condition, presence of vomiting, presence of diarrhea, etc.), information such as physical findings, medical history, examination, diagnosis, treatment/medication, prescription drug, notes, and motion video/image of an animal patient (pet). Here, the input content of the owner information, pet information, medical interview information, and pet motion video/image is comprised of data received from the user terminal 200, physical findings and other information relating to the diagnosis of the animal patient can be comprised of data received from the animal hospital terminal 300, and can be comprised of data received by the person in charge such as the doctor at the animal hospital, by inputting it into the information to the animal hospital terminal 300 based on the information obtained from the owner of the animal patient. Here, prescription drug information may include prescription drug contents, medication method of administering the drug (including schedule), contents for learning the medication method (e.g., images, illustrations, texts, motion videos, etc.), and information on the status of the drug administration, including whether the owner or other user of the animal patient has completed the administration. Further, as will be described later, the range of medical chart information that can be shared with a user such as an owner of an animal patient (e.g., limited to physical findings and examinations, etc.) can be set in response to a setting request from the animal hospital terminal 300.

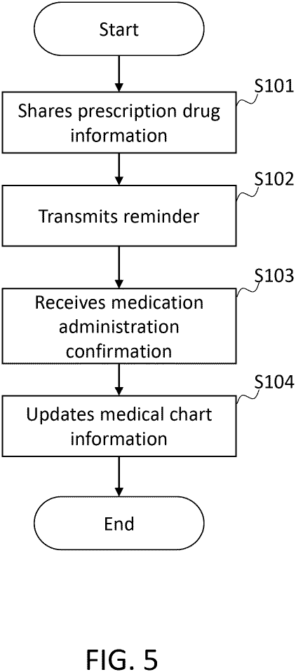

FIG. 5 is an example of a flowchart which explains a method for providing an electronic chart according to the first embodiment of the present invention.

First, prior to the processing shown in this flowchart, the instruction reception unit 131 of the control unit 130 of the server terminal 100 receives an electronic medical chart sharing request from the user terminal 200 via the communication unit 110. In addition, medical information can be shared with the user terminal 200 irrespective of the sharing request, but since it is information that requires careful handling of information related to electronic medical chart, it is preferable to share electronic medical chart information by synchronizing the electronic medical chart information stored in the server terminal 100 with the user terminal 200 in response to a sharing request from the user terminal 200. In response to the electronic medical chart information sharing request, the medical chart information management unit 132 of the control unit 130 acquires the electronic medical chart information from the electronic medical chart information storage unit 121 of the storage unit 120, and the medical chart information sharing unit 133 of the control unit 130 transmits the edited medical chart information (or related to the set range) to the user terminal 200 to share the medical chart information.

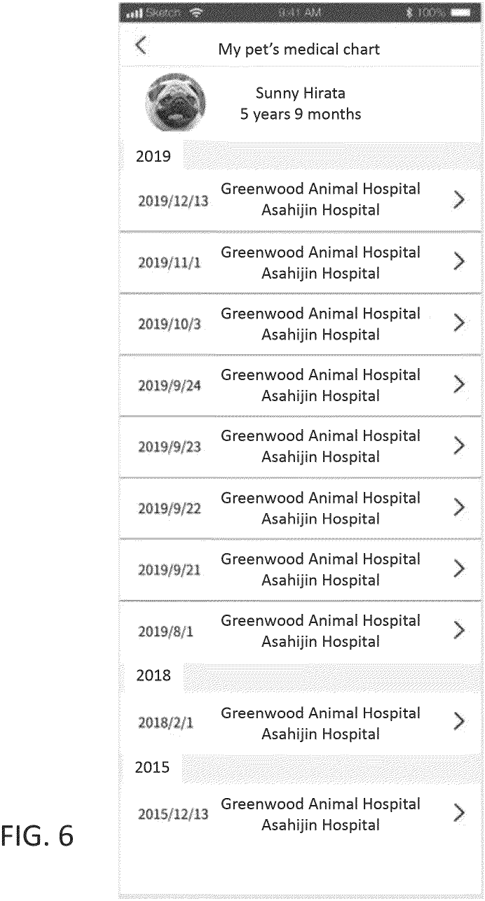

For example, as shown in FIG. 6, a list of date-by-date electronic medical chart information of target animals is displayed on the chat user interface screen of the user terminal 200. Here, the chat user interface screen may be an interface screen displayed via a chat application provided by a service operator provided by the server terminal 100 or a third party. Here, when sharing electronic medical chart information via a chat application provided by a third party, the server terminal 100 can also provide information displayed on the interface screen via an API (Application Programming Interface) of a third party’s application.

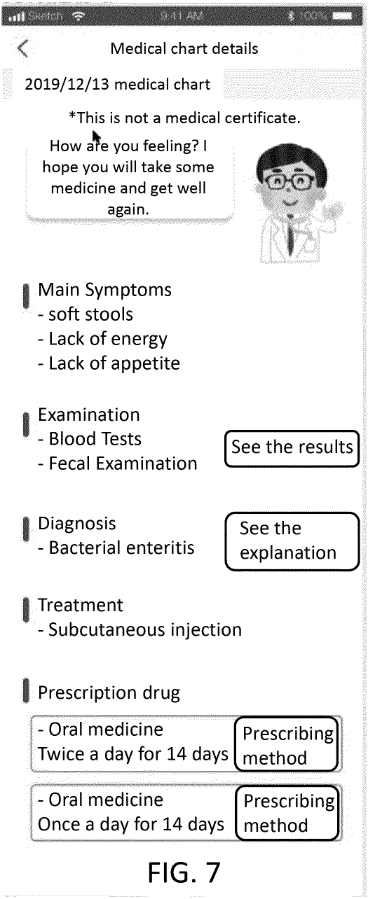

In FIG. 6, the user of the user terminal 100 selects a desired electronic medical chart, thereby displaying a detailed screen of the electronic medical record as shown in FIG. 7. As shown in FIG. 7, as the details of the electronic medical chart, main symptoms of the animal, the animal’s main symptoms, examination contents, diagnosis contents, treatment contents, prescription drug contents, etc. are displayed in addition to the opinion of the veterinarian. Here, for the contents of the examination, details of the examination results, for the diagnosis, an explanation of the diagnosis, and for the prescription drugs, the prescription method is displayed upon further request from the user terminal 100. If necessary, the user of the user terminal 100 can ask a question to the veterinarian through the chat user interface screen. Thereby, the user can refer to the electronic medical record by a simple method even when the user is located outside the animal hospital for the pet, such as at home, and thus can check the health condition of the pet at a desired timing and try to take care of it.

Here, before sharing the electronic medical chart information, the medical chart information sharing unit 133 of the control unit 130 stores the data obtained by editing the contents of the medical chart information in response to the medical chart information editing request from the animal hospital terminal 300, in the electronic medical chart information storage unit 121. For example, the veterinarian at the veterinary hospital terminal 300 deletes by editing the information about the diagnosis information in the electronic medical chart information regarding an undetermined disease or deletes a memo for the veterinarian, and then sends a file containing the edited medical chart information to the server terminal 100 or accesses the server terminal 100 and edits the medical chart information on the screen for editing the electronic medical chart information displayed on the browser of the animal hospital terminal 300. The edited electronic medical chart information is stored in the electronic medical chart information storage unit 121. Further, instead of editing the electronic medical chart information, or in advance, the range shared from the animal hospital terminal 300 to the user terminal 100 among the electronic medical chart information may be set by designating each item of the electronic record information.

Referring to FIG. 5, first, the medical chart information sharing unit 133 of the control unit 130 transmits and shares prescription drug information to the user terminal 200 (S101). Here, as described above, the medical chart information sharing unit 133 can transmit prescription drug information together with other information as medical chart information, or it can transmit only prescription drug information.

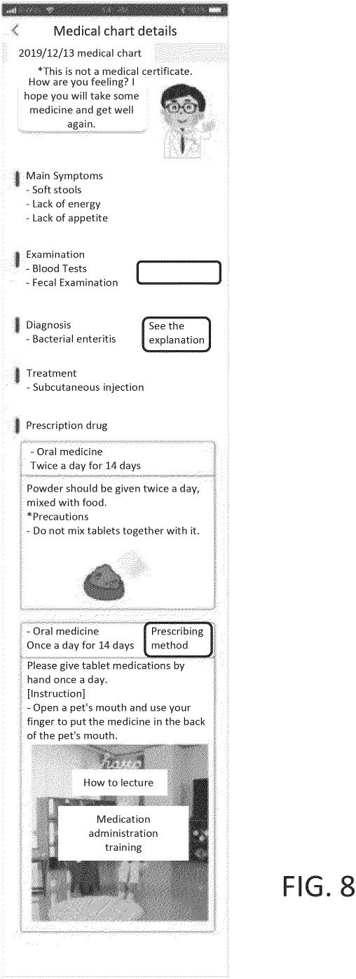

As an example, as shown in FIG. 8, prescription drug information can be shared and displayed as part of the medical chart information on the chat user interface screen of the user terminal 200. As the prescription drug information, information relating to prescription drugs includes a description that explains the frequency, period, and method of administering the medication for each drug. For example, in the example of FIG. 8, for an oral medication (tablet), the dosing frequency and period are displayed as once a day for 14 days, and for the medication method, “please open the mouth and put the medicine in the back of the mouth with a finger” is displayed, and further, a link to a motion video for performing medication training is displayed. In addition, a medication schedule (morning, noon, night, before/after meals, time if time is specified) can also be displayed. By selecting the video link, the user can learn how to administer medication to the pet while watching the motion video. In addition to motion videos, text, illustrations, images, and the like can be used as content for learning medication methods.

Further, the medical chart information sharing unit 133 of the control unit 130 refers to the electronic medical chart data 1000 stored in the electronic medical chart information storage unit 121 to check the medication schedule of prescription drugs, and send the information relating to the medication schedule and/or medication reminders to the user terminal 200 (S102). Here, the medical chart information sharing unit 133 can transmit a reminder prior to the dosing timing or medication time according to a predetermined schedule.

Next, the instruction reception unit 131 of the control unit 130 can receive a response confirming that the medication has been administered from the user terminal 200 (S103). The user of the user terminal 200 administers medication to the pet according to the above-mentioned motion video or the like, and a response to the effect, for example, that the medication has been administered by selecting the “medication administration complete” button on the chat user interface screen, can be transmitted to the server terminal 100.

Based on the received response information, the medical chart information management unit 132 of the control unit 130 updates the medication status of the prescription drug in the electronic medical chart data stored in the electronic medical chart information storage unit 121 to the effect that the medication was completed (or could not be completed), along with the time of administration, as “completed” (or updates the medication status as “not completed”) (S104). Thereby, the electronic medical chart data is updated.

As a result, even when a user is located outside a hospital, such as at a house, the administration of the medication to a pet can be performed simply.

Although embodiments according to the invention have been described above, these can be implemented in various other embodiments, and can be implemented with various omissions, replacements and changes. These embodiments and variations as well as those with omissions, substitutions and modifications are included in the technical scope of the claims and the equivalent scope thereof.

### DESCRIPTION OF REFERENCE NUMERALS

1: electronic medical chart providing system
100: server terminal
110: communication unit
120: storage unit
130: control unit
200: user terminal
300: animal hospital terminal
NW: network

1. A method for providing an electronic medical chart related to an animal patient, the method executed by a computer, comprising:
managing medical chart information, including prescription drug information regarding prescription drug contents and medication administration information for an animal patient; and
transmitting the prescription drug information to an animal patient terminal of the animal patient,
wherein the prescription drug information includes a medication administration method concerning the prescription drug and a motion video for learning the medication administration method concerning the prescription drug.

2. (canceled)

3. The method for providing an electronic medical chart according to claim 1, further comprising:
transmitting a reminder based on a medication schedule included in the prescription drug information.

4. The method for providing an electronic medical chart according to claim 1, further comprising:
receiving a response to the effect that medication administration has been completed from the animal patient terminal.

5. The method for providing an electronic medical chart according to claim 1, further comprising:
updating information relating to medication status included in the prescription drug information based on the response that medication administration has been completed.
